(19) United States
(12) Patent Application Publication
(10) Pub. No.: US 2025/0236825 A1
(43) Pub. Date: Jul. 24, 2025

(54) SYSTEMS AND METHODS FOR CO-CULTURE IN MICROPLATES

(71) Applicants: MOLECULAR DEVICES (AUSTRIA) GMBH, Puch Bei Hallein (AT); MOLECULAR DEVICES LLC, San Jose, CA (US)

(72) Inventors: Josef Atzler, Hallein (AT); Felix Spira, Neumarkt am Wallersee (AT); Sara Sofia Deville, Salzburg (AT); Oksana Sirenko, San Carlos, CA (US)

(21) Appl. No.: 18/848,410

(22) PCT Filed: Mar. 14, 2023
(86) PCT No.: PCT/US2023/064313

§ 371 Date: Sep. 18, 2024

Related U.S. Application Data
(60) Provisional application No. 63/322,058, filed on Mar. 21, 2022.

(51) Int. Cl. C12M 3/00; C12M 1/00; C12M 1/12; C12M 1/26; C12N 5/00

(52) U.S. Cl. CPC C12M 21/08; C12M 23/58; C12M 25/04; C12M 33/04; C12N 5/0062

(57) ABSTRACT

A culture plate includes an upper surface and a plurality of well systems that include a first well, a second well, a first channel in fluid communication with the first and second wells, and a third well between the first and second wells, the third well being in fluid communication with the first channel, the upper surface of the culture plate defining a well opening for each of the first, second, and third wells. A method of cell separation in a culture plate includes loading a liquid hydrogel inside a lower chamber, solidifying the hydrogel, loading the first well and/or the second well with cells, spheroids and/or organoids, introducing the cells, spheroids and/or organoids in the first channel to allow an interaction thereof with the hydrogel, and adding feeding media to the first, second and/or third wells.

### CROSS-REFERENCE TO RELATED APPLICATIONS

This application is the national phase of PCT International Patent Application No. PCT/US2023/064313 filed on Mar. 14, 2023, which in turn claims priority to and the benefit of U.S. Provisional Application No. 63/322,058, filed on March 21. 2022, the disclosure of which is hereby incorporated by reference in its entirety.

### BACKGROUND

Co-culturing enables the development of artificial microbial communities to demonstrate the competition between microorganisms within the same environment. Co-culturing also allows a variety of cell types to be cultured together to examine the effect of one culture system on another, which is useful when examining the effect of one type of tissue on another, one region of the brain on another, or how a particular secreted molecule leads to changes in neural development or physiology. For example, co-cultures of different regions of spinal cord explants may reveal differing effects on the ability to attract or repel neurite outgrowth, or biochemical purification from explants in co-culture experiments may lead to the identification of specific molecules that can then be introduced into cell lines to express and secrete molecular guidance cues.

### SUMMARY

In one aspect, the technology relates to a culture plate that includes an upper surface, and a plurality of well systems, each well system including a first well, a second well, a first channel in fluid communication with the first well and with the second well, and a third well disposed between the first well and the second well, wherein the third well is in fluid communication with the first channel, wherein the upper surface of the culture plate defines a well opening for each of the first well, the second well, and the third well. In an example, the first channel is in fluid communication with the first well on a side on the first well and with the second well on a side on the second well. In another example, the third well includes an upper chamber and a lower chamber having a diameter less than a diameter of the upper chamber, wherein the first channel is in fluid communication with the lower chamber. In further examples, the first well and the second well are in fluid communication with the first channel via a first opening and a second opening, respectively; the first channel is a microchannel having an inside diameter of about 5 μm to 100 μm; an inside diameter of the first channel comprises a collagen coating; a profile of the inside diameter of the first channel is one of circular, oval, and polygonal; the first channel extends horizontally between the first well and the second well; the first channel extends non-linearly between the first well and the second well.

In an example of the above aspect the culture plate further includes a fourth well, a fifth well, and a second channel in fluid communication with the third well, the fourth well and the fifth well. In a further example, the culture plate includes one of 96 well plates and 348 well plates.

In another aspect, the technology relates to a method of cell separation from a media flow in a culture plate comprising a plurality of well plates, each well plate comprising at least a first well, a second well, a third well and a first channel in fluid communication with the first well, the second well and the third well, the third well including an upper chamber and a lower chamber, the method including loading a liquid hydrogel inside the lower chamber, solidifying the hydrogel via incubation, loading at least one of the first well and the second well with at least one of cells, spheroids and organoids, introducing the at least one of cells, spheroids and organoids in the first channel from the at least one of the first well and the second well to allow an interaction thereof with the hydrogel, and adding feeding media to the at least one of the first well, the second well and the third well. In an example, the interaction includes at least one of cell adherence and organoid formation. In a further example, introducing the at least one of cells, spheroids and organoids in the first channel includes sealing an opening of at least one of the first well and the second well with a pipette. In other example, the method further includes accessing at least one of the first well, the second well and the third well via an opening therein to manipulate contents thereof; the method further includes removing air from the first channel before adding the feeding media; and loading the hydrogel inside the lower chamber comprises transferring the hydrogel from the upper chamber into the lower chamber.

In an example of the above aspect, the culture plate further includes a second channel in fluid communication with the third well, a fourth well and a fifth well, the method further includes loading at least one of the fourth well and the fifth well with at least one of cells, spheroids and organoids, and adding feeding media to at least one of the fourth well and the fifth well. In another example, the method further includes adding epithelial cells to the upper chamber, wherein loading the at least one of the first well and the second well comprises loading the at least one of the first well and the second well with at least one of organoids and tumoroids. In a further example, the method further includes adding differentiating media to the upper chamber, adding precursor cells to the at least one of the first well and the second well, and imaging neuron healing from the opening in the upper chamber. In yet another example, loading the liquid hydrogel includes loading the liquid hydrogel with a cell repelling hydrogel, and the method further includes maintaining the culture plate at a desired temperature to allow the hydrogel to flow to the first channel before the incubation, and filling the first channel with hydrogel mixed with at least one of cells, spheroids and organoids.

In another example of the above aspect, loading the liquid hydrogel includes loading a cell repelling hydrogel, and the method further includes maintaining the culture plate at a desired temperature to allow the hydrogel to flow to the first channel before the incubation, and filling the first channel with media mixed with cells capable of forming tub vessels. In a further example, loading the liquid hydrogel includes loading the liquid hydrogel with a cell repelling hydrogel, and the method further includes maintaining the culture plate at a desired temperature to allow the hydrogel to flow to the first channel before the incubation, and filling the first channel with media mixed with cells capable of forming a lung tissue or epithelium. In yet another example, the method includes maintaining the incubation until the cells are settled at a bottom portion of the upper chamber and the lung epithelium or tissue is formed.

In a further example of the above aspect, loading the liquid hydrogel and loading at least one of the first well and the second well include loading a different combination of cells, spheroids and organoids in each of the first well, the second well and the third well, and the first well, the second well and the third well are subsequently fluidly connected by removing air present inside the first channel. In another example, removing the air comprises one of using a vacuum and scalingly engaging a pipette tip to aspirate the air from the first channel. In a further example, loading the liquid hydrogel, loading at least one of the first well and the second well, and loading at least one of the fourth well and the fifth well include a different combination of cells, spheroids and organoids in each of the first well, the second well, the third well, the fourth well and the fifth well, the first well, the second well and the third well are fluidly connected by removing air present inside the first channel, and the third well, the fourth well and the fifth well are fluidly connected by removing air present inside the second channel. In a further example, loading the liquid hydrogel includes loading a liquid hydrogel premixed with at least one of cells, spheroids and organoids in the first channel via a loading port in the third well by sealingly engaging a pipette with the premixed hydrogel at the loading port. As another example, loading the liquid hydrogel includes loading a liquid hydrogel premixed with at least one of cells, spheroids and organoids in the first channel and in the second channel via a loading port in the third well by sealingly engaging a pipette with the premixed hydrogel at the loading port. In a further example, loading the liquid hydrogel includes loading a liquid hydrogel premixed with at least one of cells, spheroids and organoids in the first channel via a loading port in the third well by sealingly engaging a pipette with the premixed hydrogel at the loading port, and loading the feeding media in the second channel.

These and other advantages, aspects and novel features of the present disclosure, as well as details of an illustrated embodiment thereof, will be more fully understood from the following description and drawings.

### BRIEF DESCRIPTION OF THE DRAWINGS

FIGS. 1A-1C are illustrations of a microplate arrangement, in accordance with various examples of the disclosure.

FIGS. 2A-2B are illustrations of a microplate array, in accordance with various examples of the disclosure.

FIGS. 3A-3C are illustrations of various operations of microplate arrangements, in accordance with various examples of the disclosure.

FIGS. 4A-4D are illustrations of a microplate arrangement, in accordance with various examples of the disclosure.

FIGS. 5A-5G are examples of various operations of a microplate arrangement, in accordance with various examples of the disclosure.

FIG. 6 is a flowchart illustrating a method of separating cells, in accordance with an example of the disclosure.

FIG. 7 depicts a block diagram of a computing device, in accordance with an example of the disclosure.

### DETAILED DESCRIPTION

The current state of product development, and scientific advancement in general, for example in the life sciences, is challenged by existing systems and methods which delay product and/or scientific development cycles. For example, separating cells and cell aggregates from a media flow by a hydrogel barrier, and simultaneously maintaining direct access for mechanical manipulation of the separated cells or aggregates, is typically challenging. It is typically difficult to simultaneously monitor and mechanically manipulate bundles of neurons or muscle tissue or other growing structures. In addition, multi-organ systems are difficult to generate in a microplate format, and, e.g., tube-wired microfluidic chips are often used.

Accordingly, a technical problem exists, the technical problem being the ability to separate cells and cell aggregates from a media flow by a hydrogel barrier in order to contemporaneously maintain direct access to the cells and/or cell aggregates for mechanical manipulation of the separated cells or aggregates for, e.g., experimental purposes. One solution to this technical problem may include connecting two wells with a channel and introducing an opening into the center of the channel to allow access for manipulation of cells in the channel. For example, the opening may have structural elements that allow the formation of a hydrogel barrier that separates the medium of a feeding well from the medium inside the channel. Accordingly, by having an opening of the central channel that can be accessed from the top, it becomes possible to manipulate cells in this well.

In various examples of the disclosure, seeding cells into a central well and monitoring the outgrowth of the cells into a channel linking two wells, or monitoring the outgrowth from one side channel towards the central channel, may make it possible to e.g., sever or manipulate the cell, or introduce measurement probes into the central channel. Adding multiple channels may allow for more cells to be seeded and monitored, and may also increase the complexity of the monitored system. For example, in order to create multi-organ system, different types of cells, spheroids or organoids may be seeded into one of the three different wells, a feeding well, a culture well and a central well connected to a central channel. By creating one of multiple barriers, complex organ combinations such as, e.g., kidney, blood-brain barrier, or liver may be replicated and studied. This may be useful for studies such as, e.g., toxicology studies, or brain studies. The accessibility of the central well may also allow for the creation of, e.g., lung systems, by having one side exposed to ambient air, while the other side is exposed to media. Thus, given the versatility of the microplate, the microplate may be used in a number of various applications other than the applications discussed above.

It will be understood that, although the terms first, second, etc. may be used herein to describe various elements, these elements should not be limited by these terms. These terms are only used to distinguish one element from another element. Thus, for example, a first element, a first component or a first section discussed below could be termed a second element, a second component or a second section without departing from the teachings of the present disclosure. Similarly, various spatial terms, such as “upper,” “lower,” “side,” and the like, may be used in distinguishing one element from another element in a relative manner. It should be understood, however, that components may be oriented in different manners, for example a lifting device may be turned sideways so that its “top” surface is facing horizontally and its “side” surface is facing vertically, without departing from the teachings of the present disclosure.

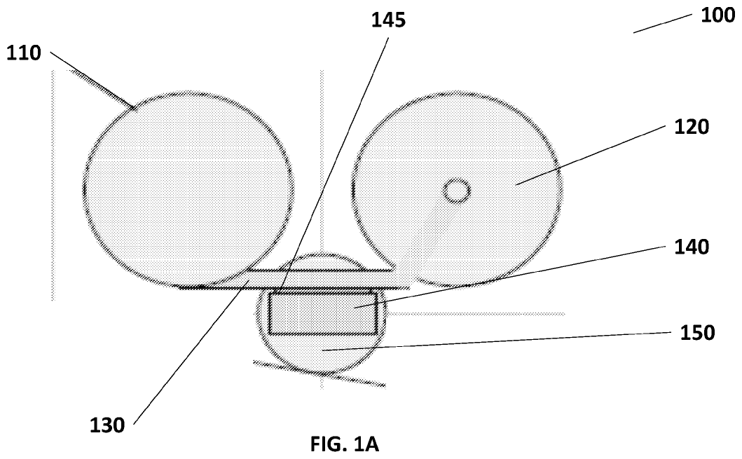

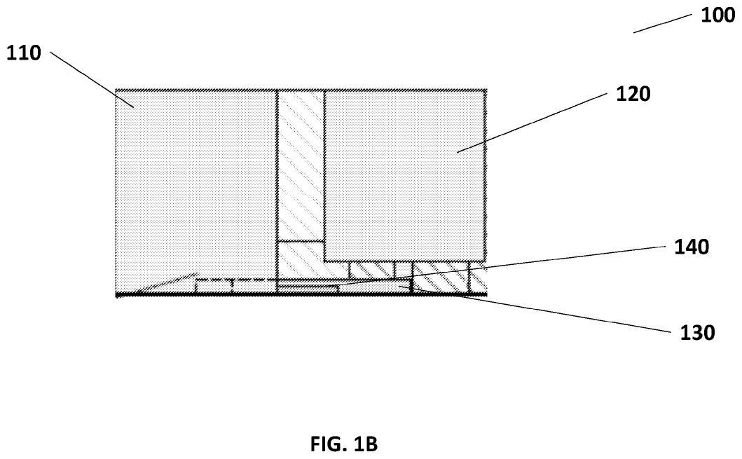

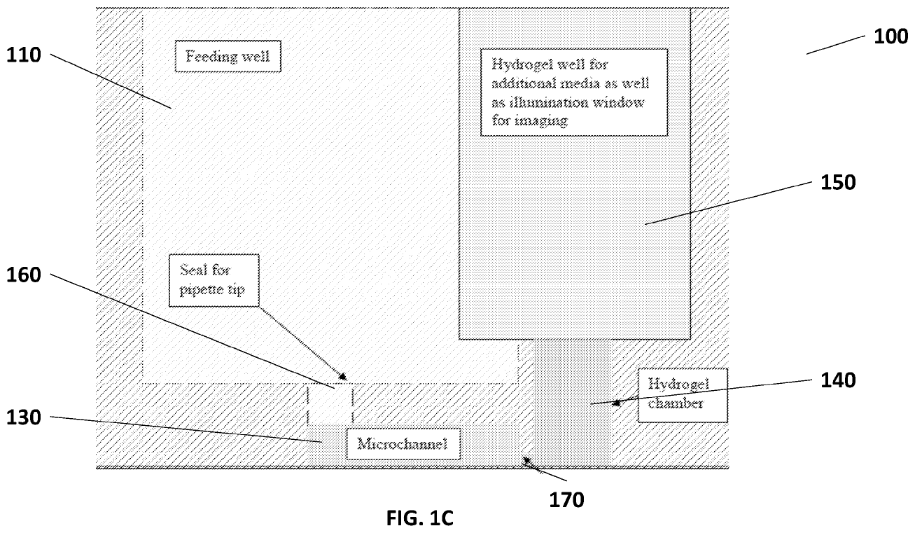

FIGS. 1A-1C are illustrations of a microplate arrangement, in accordance with various examples of the disclosure. In FIGS. 1A and 1B, the microplate arrangement 100 includes a culture well 120 and a feeding well 110. For example, the feeding well 110 is a well that may hold cells or cell aggregates, and may be referred to herein as the first well. The culture well 120, also referred to herein as the second well, and the feeding well 110 may be connected via a channel 130 such as, e.g., a microchannel. In an example, the channel 130 is a microchannel having an inside diameter of about 5 μm to 100 μm. In another example, an inside diameter of the channel 130 has a coating of one or more matrix proteins such as, e.g., a structural protein such as collagen. In yet another example, a profile of the inside diameter of the channel 130 may have different shapes such as, e.g., circular, oval, or polygonal. In a further example, the channel 130 extends horizontally between the feeding well 110 and the culture well 120. The channel 130 may also extend non-linearly between the feeding well 110 and the culture well 120 by having a curvature configured to support, e.g., the formation intestinal organoids, or the formation of other types of organoids or spheroids therein. In various examples, the channel 130 may be connected to a hydrogel well 150 via a hydrogel chamber 140. For example, the hydrogel well 150, also referred to herein as the third well, may constitute an upper chamber of the third well, and the hydrogel chamber 140 may constitute the lower chamber of the third well. In another example, the hydrogel well 150 and hydrogel chamber 140 may be unconnected to either the culture well 120 or the feeding well 110 except via the channel 130. In an example, the hydrogel chamber 140 includes a liquid phase guide 145. For example, the liquid phase guide 145 may be a ridge formed in the hydrogel chamber 140 to receive the channel 130.

FIG. 1C is a side view of the microplate arrangement illustrated in FIGS. 1A-1B, according to various examples of the disclosure. In FIG. IC, the microplate arrangement 100 includes the feeding well 110 fluidly connected to the channel 130. In various examples of the disclosure, the connection channel 130 is fluidly connected to the feeding well 110 via an opening 160 formed at a bottom portion of the feeding well 110. For example, the opening 160 may have a shape and size such that a typical pipette may be able to seal it by substantially covering the opening 160. In another example, the feeding well 110 may be of a shape and size such that a pipette may form a seal by contacting an upper rim thereof. In various examples, the hydrogel well 150 may include additional media for the hydrogel chamber 140. In other examples, the microplate arrangement 100 includes an interface 170 between the channel 130 and the hydrogel chamber 140. At the interface 170, the hydrogel present in the hydrogel chamber 140 may not be able to enter the channel 130 due to, e.g., the shape and/or profile of an interior cavity of the channel 130. However, the media present in the hydrogel chamber 140 may interact with the cells and/or aggregates present in the channel 130. In another example, each of the feeding well 110, the culture well 120 and the hydrogel well 150 or hydrogel chamber 140 may include a different combination of cells, spheroids, and organoids.

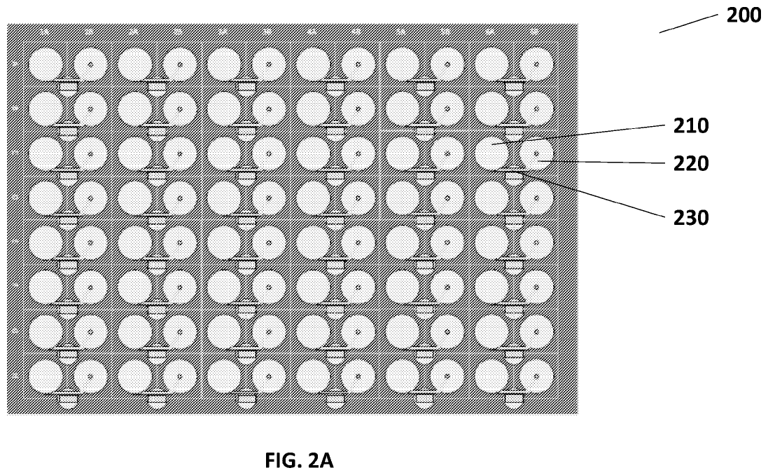

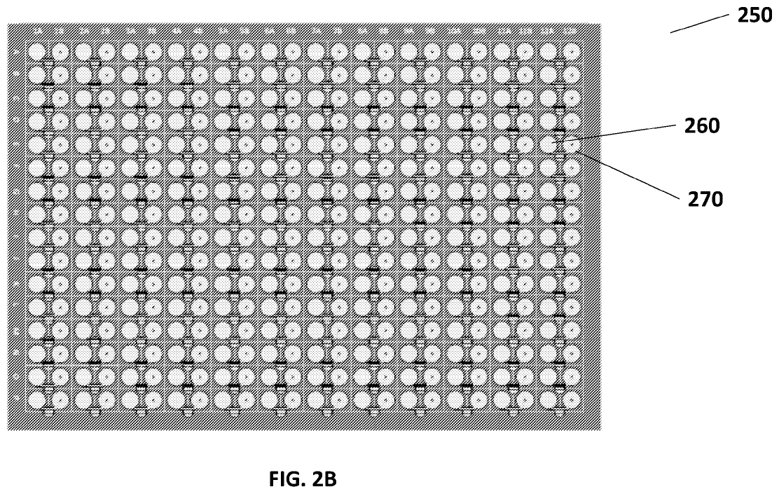

FIGS. 2A-2B are illustrations of microplate arrays 200 and 250, in accordance with various examples of the disclosure. In FIG. 2A, the microplate array 200 includes 48 microplate arrangements, each microplate arrangement including a culture well 220 and a feeding well 210 fluidly connected with a channel 230, each microplate arrangement being similar to the microplate arrangement 100 discussed above with respect to FIGS. 1A-1C. Accordingly, there is a total of 96 wells in the microplate arrangement 200 illustrated in FIG. 2A. In FIG. 2B, the microplate array 250 includes 192 microplate arrangements, each microplate arrangement including a feeding well 260 and a culture well 270 connected with a channel, each microplate arrangement being similar to the microplate arrangement 100 discussed above with respect to FIGS. 1A-IC. Accordingly, there is a total of 384 wells in the microplate arrangement 250 illustrated in FIG. 2B.

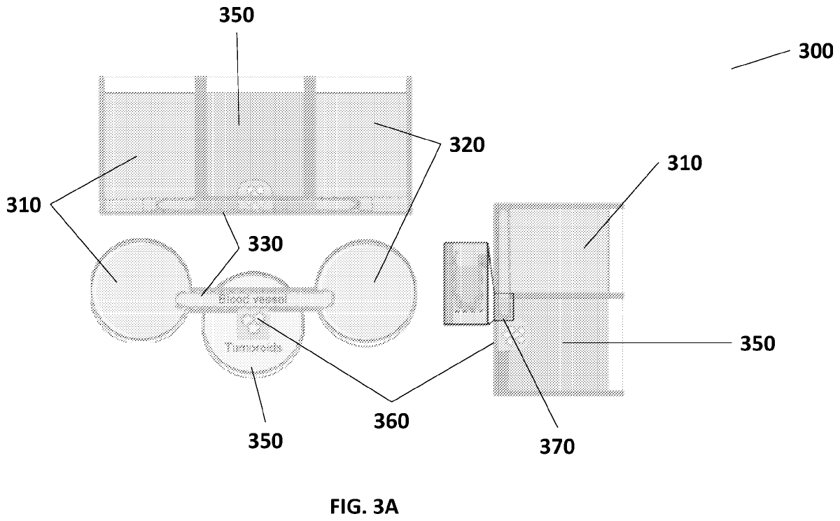

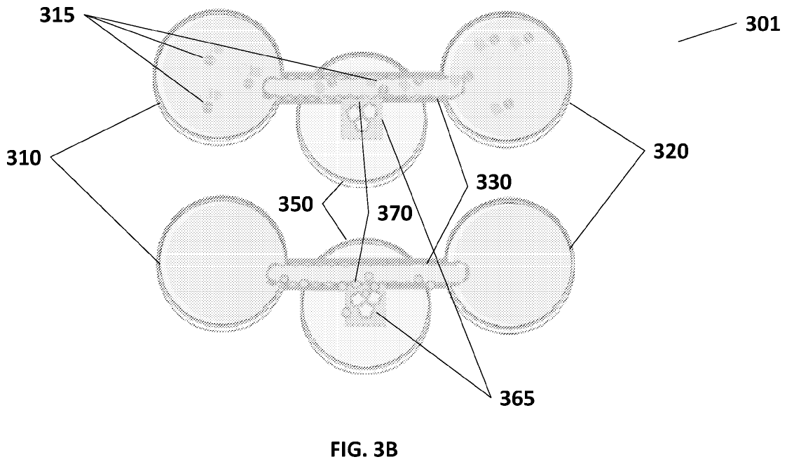

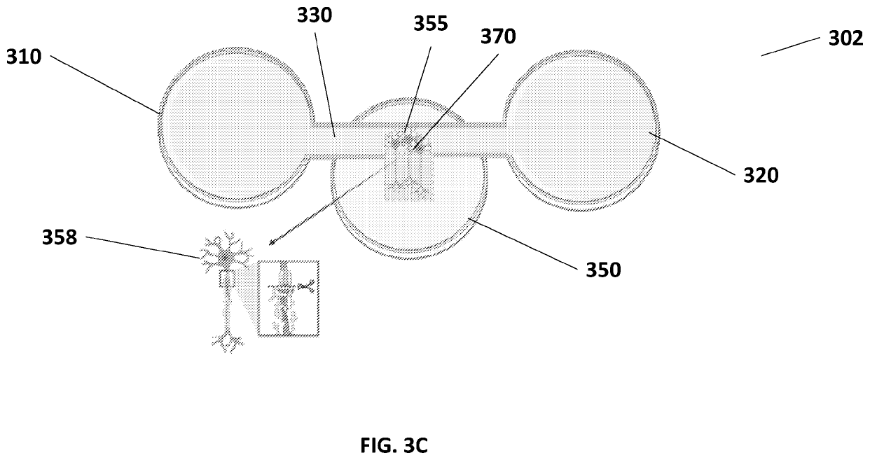

FIGS. 3A-3C are illustrations of various operations of microplate arrangements, in accordance with various examples of the disclosure. In FIG. 3A, the microplate arrangement 300 is a blood brain barrier/gut barrier arrangement that includes a culture well 320 and a feeding well 310 fluidly connected with a channel 330. In operation, a hydrogel may be loaded in the hydrogel well 350, the hydrogel being mixed with, e.g., cells, spheroids and/or organoids. In various examples, the hydrogel may be hardened by, e.g., heating at a desired temperature, and a medium mixed with cells such as, e.g., epithelial cells, may be added to the feeding well 310. For example, the medium may be pushed to the bottom of the feeding well 310 via, e.g., a pipette, so that the medium mixed with cells may travel through the channel 330 and up to the interface 370 between the channel 330 with the hydrogel chamber 350. Specifically, the channel 330 may be filled by first pushing out the air trapped therein via, e.g., a pipette. Alternatively, the air may be removed via the application of a vacuum. As such, the medium mixed with the cells from the feeding well 310 may be pushed through the channel 330 towards the culture well 320. Accordingly, the cells and the medium that are now inside the channel 330 may form a vessel-like structure and may interact with cells, spheroids and/or organoids present in the channel 330 at the interface 370. As a result, the culture well 320, the feeding well 310 and the channel 330 are now fluidically connected, and a complex system with a barrier at the interface 370 between the channel 330 and the hydrogel well 350 may be reproduced and modelled. In various examples, the model thus created may be used to evaluate, e.g., vessel sprouting and branching towards the organoids or tumoroids 360 that may form in the hydrogel well 350, or for tumor vascularization.

In FIG. 3B, in various examples of the disclosure, the microplate arrangement 301 is a blood brain barrier/gut barrier arrangement that includes the culture well 320 and the feeding well 310 fluidly connected to each other via the channel 330. In operation, the hydrogel in the hydrogel well 350 may be mixed with, e.g., cells, spheroids and/or organoids, and a medium mixed with cells such as, e.g., epithelial cells 315 may be added to the feeding well 310. For example, the medium and epithelial cells 315 may be pushed through the channel 330, as discussed above with respect to FIG. 3A, and travel at the interface 370 between the channel 330 and the hydrogel chamber 350. As such, the cells 315 and the medium that are inside the channel 330 may form a vessel-like structure, and a complex system with a barrier at the interface 370 between the channel 330 and the hydrogel well 350 may be modelled. In various examples, the model thus created may be used to evaluate vessel sprouting and branching towards the organoids or tumoroids 365 that may form in the hydrogel well 350. The example illustrated in FIG. 3B may allow, e.g., testing of compound permeability or immune cell infiltration and extravasation.

In FIG. 3C, in various examples of the disclosure, the microplate arrangement 302 mimics a neurite outgrowth, ablation and healing or recovery evaluation arrangement. In FIG. 3C, the culture well 320 and the feeding well 310 are fluidly connected to each other via the channel 330. In operation, a differentiation media may be added and mixed with the hydrogel in the hydrogel well 350, and precursor cells 355 may be added to the medium in the feeding well 310. Accordingly, the precursor cells 355 may pushed or travel from the feeding well 310 to the channel 330 and close to, or against, the interface 370 between the channel 330 and the hydrogel chamber 350. As discussed above with respect to FIG. IC, in operation, the medium mixed with the precursor cells to the feeding well 310 may be added by sealing a pipette tip containing the mixture to the bottom of the feeding well 310. Once the mixture is added to the feeding well 310, the channel 330 may be filled by removing any air present therein and allowing the mixture of medium and cells 355 to settle in the channel 330. Accordingly, the feeding well 310, the culture well 320 and the channel 330 are fluidly connected, and neurons 358 may grow from the precursor cells 355 towards the differentiation media present in the hydrogel chamber 350. Accordingly, once a certain growth of the neurons 358 is reached, experimentation may be carried out on the neurons 358. For example, damage to the neurons 358 may be induced, or portions of the neurons 358 may be excised, or the like, in order to observe, e.g., the healing or recovery process of the neurons 358 post-damage. In various examples, observation of the healing or recovery process may be carried out due to the existence of an observation window over the hydrogel chamber 350.

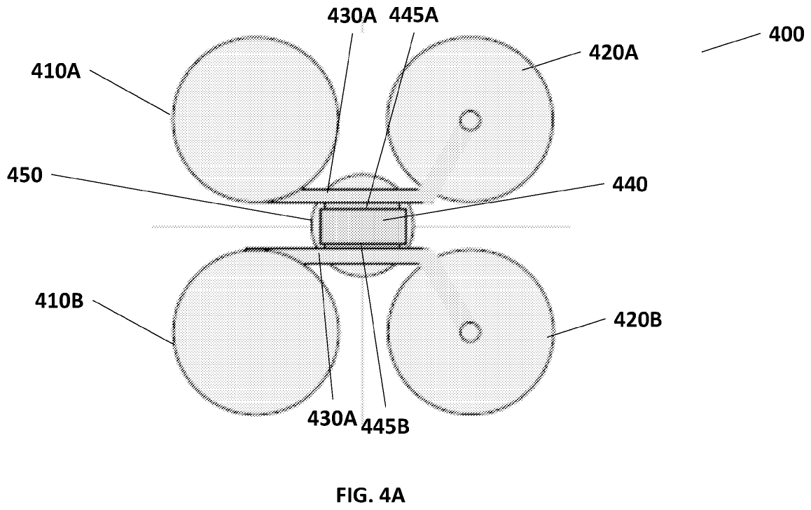

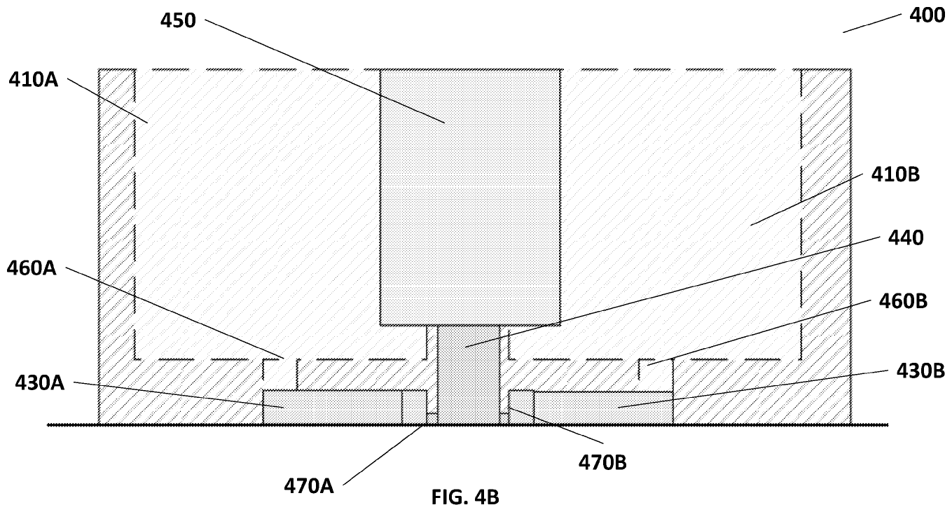

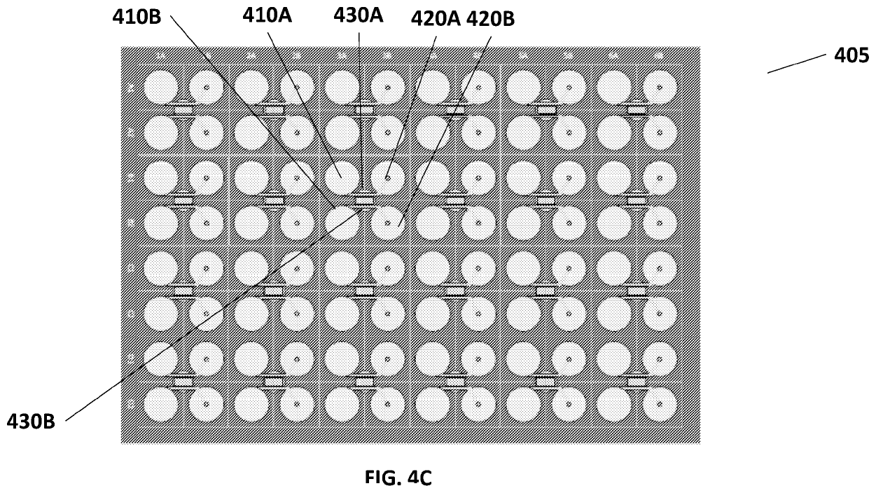

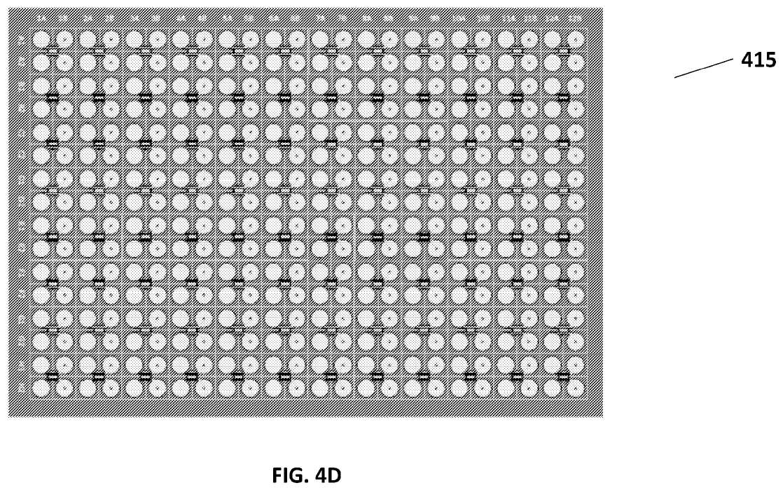

FIGS. 4A-4D are illustrations of a microplate arrangement, in accordance with various examples of the disclosure. In FIG. 4AB, the microplate arrangement 400 includes two feeding wells 410A and 410B, two culture wells 420A and 420B, and two channels, or microchannels, 430A and 430B. For example, feeding well 410A, culture well 420A and channel 430A are fluidly connected to each other, and feeding well 410B, culture well 420B and channel 430B are fluidly connected to each other. For example, feeding well 410B, culture well 420B and channel 430B are fluidly unconnected to any of feeding well 410A, culture well 420A or channel 430A. In various examples of the disclosure, both microchannels 430A and 430B are in fluid communication with a same hydrogel chamber 440 and hydrogel well 450. Accordingly, the feeding wells 410A and 410B may hold cells, precursor cells, or aggregates, and feeding well 410A may hold cells or aggregates that are different from the cells or aggregates held in feeding well 410B. In various examples, the media in the feeding wells 410A and 410B may be independently pushed through the microchannels 430A and 430B respectively, so as to be able to interact with any media such as, e.g., feeding media, mixed with the hydrogel chamber 440. As both the microchannels 430A and 430B are in contact with the same hydrogel well 440, the cells in each of the microchannels 430A and 430B may interact with the same media or precursor cells present in the hydrogel well 440. As such, two different types of interactions may be contemporaneously or simultaneously created and observed, one between the contents of the microchannel 430A and the media present in the hydrogel well 440, and the other between the contents of the microchannel 430B and the media present in the hydrogel well 440. In an example, the hydrogel well 440 includes liquid phase guides 445A and 445B. For example, the liquid phase guide 445A may be a ridge formed in the hydrogel well 440 to receive the microchannel 430A, and the liquid phase guide 445B may be a ridge formed in the hydrogel well 440 to receive the microchannel 430B.

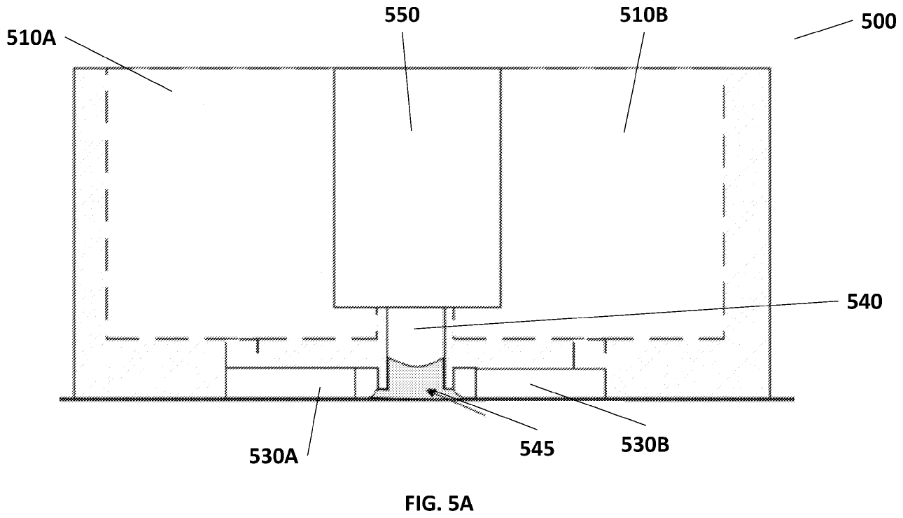

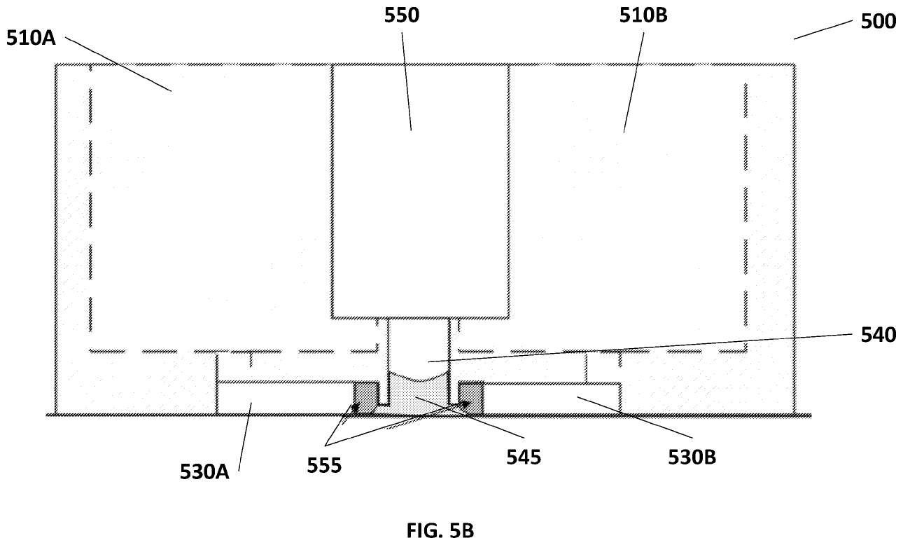

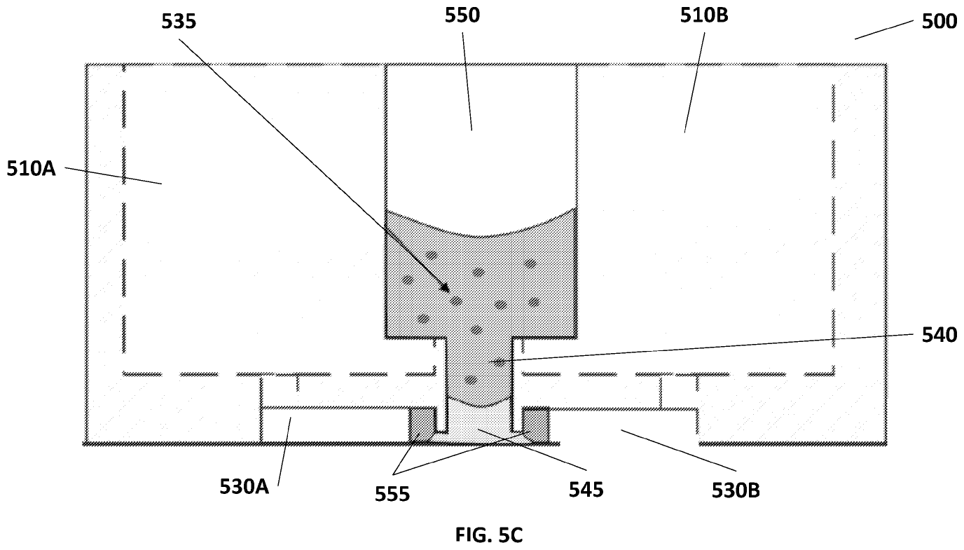

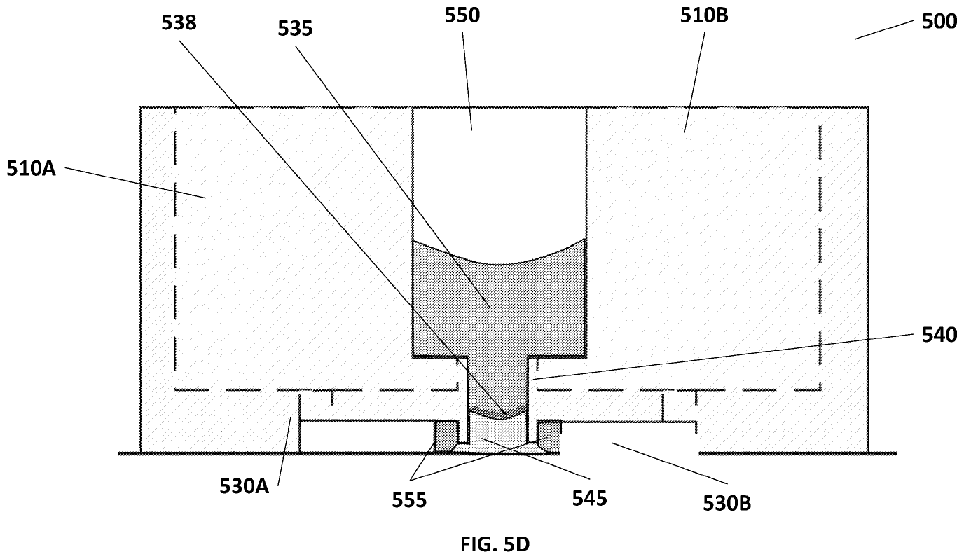

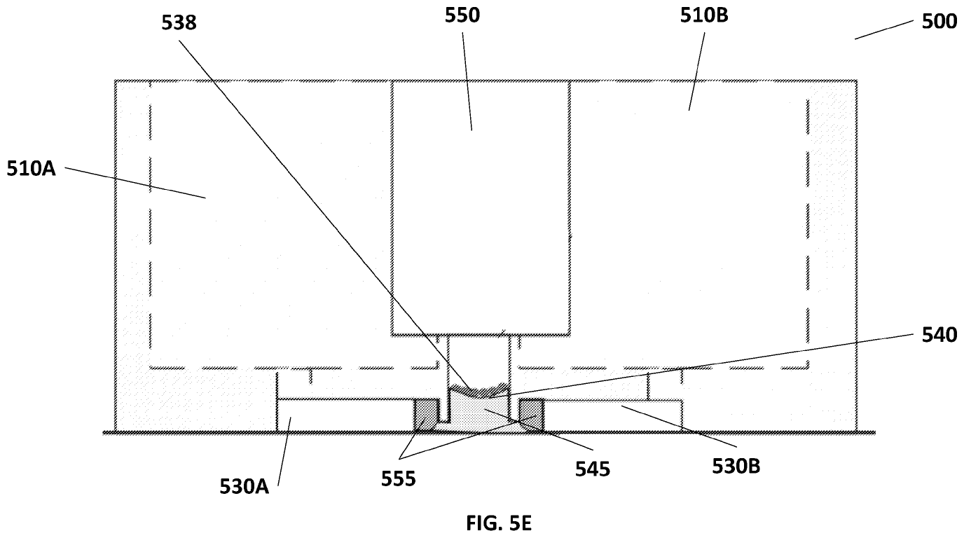

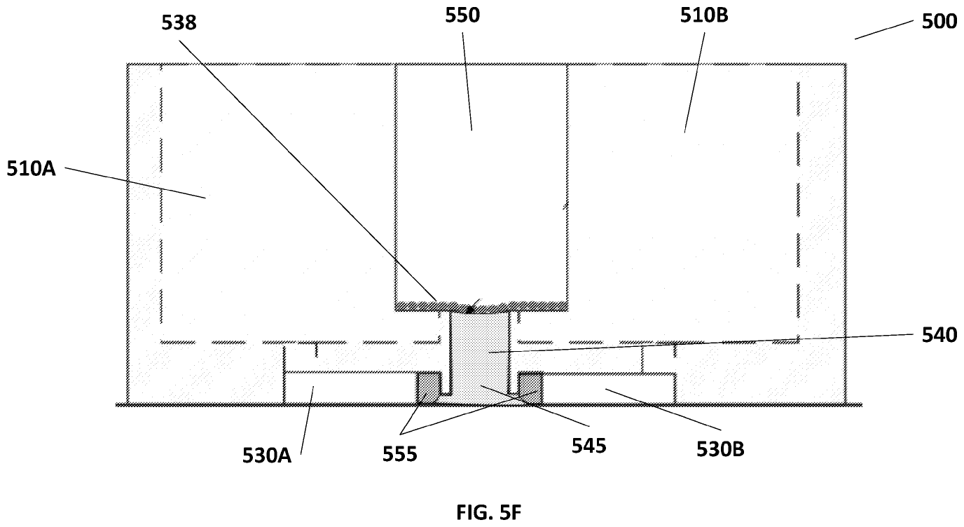

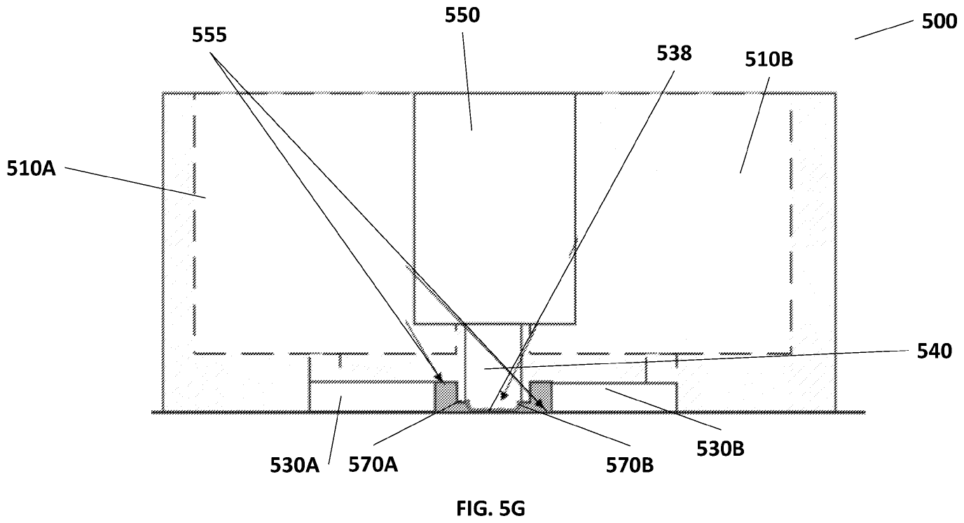

FIG. 4B is a side view of the microplate arrangement 400 illustrated in FIG. 4A, according to various examples of the disclosure. In FIG. 4B, the microplate arrangement 400 includes two feeding well 410A and 410B, fluidly connected to the microchannels 430A and 430B, respectively. In various examples of the disclosure, the microchannel 430A is coupled to the feeding well 410A via an openings 460A formed at a bottom portion of the feeding well 410A. Also, the microchannel 430B is coupled to the feeding well 410B via an openings 460B formed at a bottom portion of the feeding well 410B. For example, the openings 460A and 460B may have a size and shape such that a typical pipette may be able to seal them by substantially covering the openings 460A and 460B. Alternatively, the air inside the microchannels 430A and 430B may be a seal. In various examples, the hydrogel well 450 may include additional media for the hydrogel chamber 440, and any media present in the hydrogel chamber 440 may interact with the cells and/or aggregates present in both the microchannel 430A and the microchannel 430B and coming from the feed wells 410A and 410B, respectively. In other examples, the microplate arrangement 400 includes interfaces 470A and 470B, the interface 470A being between the microchannel 430A and the hydrogel chamber 440, and the interface 470B being between the microchannel 430B and the hydrogel chamber 440. At the interfaces 470A and 470B, the media present in the hydrogel chamber 440 may not be able to enter the microchannels 430A and 430B due to, e.g., the shape and/or profile of an interior cavity of the microchannels 430A and 430B. In an example, the interfaces 470A and 470B may also create a barrier that substantially prevents the media from entering the microchannels 430A and 430B, respectively, due to, e.g., the fact that the surface tension of the media may result in one section of the microchannels 430A and 430B being filled with media and another section of the microchannels 430A and 430B being filled with air, or with another hydrogel, as further illustrated in FIG. 5B below. The barrier created by the interfaces 470A and 470B may be a ridge of several micrometers in height. In other examples, the media present in the hydrogel chamber 440 may interact with the cells and/or aggregates present in the microchannels 430A and 430B. In another example, each of the feeding wells 410A and 410B, the culture wells 420A and 420B and the hydrogel chamber 440 may include a different combination of cells, spheroids and organoids.

FIGS. 4C-4D are illustrations of microplate arrays 405 and 415, in accordance with various examples of the disclosure. In FIG. 4C, the microplate array 405 includes 24 microplate arrangements, each microplate arrangement including two culture wells 420A and 420B, two feeding wells 410A and 410B respectively fluidly connected with two microchannels 430A and 430B via microchannels 430A and 430B, each microplate arrangement being similar to microplate arrangement 400 discussed above with respect to FIG. 4A. Accordingly, there is a total of 96 wells in the microplate arrangement 405 illustrated in FIG. 4C. In FIG. 4D, the microplate array 415 includes 96 microplate arrangements, each microplate arrangement being similar to the microplate arrangement 400 discussed above with respect to FIGS. 4A. Accordingly, there is a total of 384 wells in the microplate arrangement 415 illustrated in FIG. 4D.

FIGS. 5A-5G are examples of operations of a microplate arrangement, in accordance with various examples of the disclosure. In FIG. 5A, a microplate arrangement 500 such as, e.g., the microplate arrangement 400 illustrated in FIG. 4A above, may be part of a microplate array such as, e.g., the microplate array 405 illustrated in FIG. 4C. In this example, the microplate arrangement 500 is configured to generate a lung model, and includes feeding wells 510A and 510B fluidly connected to a hydrogel well 550 via microchannels 530A and 530B, respectively. In an example, at the bottom of the hydrogel well 550, e.g., in hydrogel chamber 540 of the hydrogel well 550, a cell repelling hydrogel 545 may be held. In operation, the microplate arrangement 500 may be kept at a desired temperature to allow the hydrogel 545 at an interface between the hydrogel chamber 540 and the microchannels 530A and 530B to solidify.

In FIG. 5B, the microplate arrangement 500 further holds a second hydrogel 555 inside a portion of the microchannels 530A and 530B which fluidly connect to the feeding wells 510A and 510B. For example, the second hydrogel 555 may be a Matrigel or replacement thereof mixed with, e.g., cells, spheroids and/or organoids. In another example, one of the microchannels 530A and 530B is filled with media mixed with cells that are capable of creating tubs similar to blood vessels. The other one of the microchannels 530A and 530B may also be filled with the same media mixed with cells capable of creating tubes similar to blood vessels, but may also be filled with other media and/or other cells and may be used, e.g., as a drug delivery medium.

In FIG. 5C, an additional medium or media 535 may be added to the hydrogel well 550. For example, the media 535 may be capable of creating a lung epithelium or tissue in the hydrogel well 550. In FIG. 5D, the microplate arrangement 500 is incubated, or maintained at a desired temperature or temperature range, until the cells of the media 535, illustrated in FIG. 5C, settle at the bottom of the hydrogel chamber 540, forming a membrane 538, as illustrated in FIG. 5D. FIG. 5D also shows that the media 535 may remain over the newly formed membrane 538. In FIG. 5E, the media 535 that remained above the membrane 538, may be removed. As a result, the membrane 538, also referred to herein as cell layer, may be exposed to ambient air in the hydrogel well 550. In various examples, in addition to epithelial cells, other different types of lung cells, may be added to create a multi-component lung system, with epithelial cells on top, and lung tissue cells underneath the epithelium cells. In another example, a thin gel layer may be added to separate both epithelial cells and tissue cells. In another example, in FIG. 5F, more hydrogel 545 may be added to, e.g., fill the hydrogel chamber 540 substantially entirely via the hydrogel well 550, so that the membrane 538 is now at the bottom of the hydrogel well 550. Accordingly, a membrane 538 is now formed at the bottom of the hydrogel well 550. With the configuration illustrated in FIG. 5F, pipetting of the media and cells in the microchannels 530A and 530B, or removing liquid form the hydrogel well 550, becomes easier. In another example, in FIG. 5G, when both microchannels 530A and 530B hold therein a hydrogel 555 such as, e.g., Matrigel or a Matrigel replacement, there may no longer be a need for a cell repelling hydrogel such as, e.g., hydrogel 545 discussed above in FIG. 5F. As a result, specific cell types such as, e.g., the cells of the membrane 538, may settle directly at the bottom of the hydrogel chamber 540, and the hydrogel well 550 may only include air so as to expose the membrane 538 directly to air. For example, the membrane 538 which settles at the bottom of the hydrogel chamber 540 may also be coated with, e.g., a 3D matrix.

In an example, the cells of the membrane 538 may also be in contact with the Matrigel or Matrigel replacement 555 present in both microchannels 530A and 530B at the interfaces 570A and 570B between the microchannels 530A and 530B and the hydrogel chamber 540.

In various examples of the present disclosure, a number of other assays, also referred to as plate reader assays, may be arranged for imaging. For example, in an angiogenesis application, a tumor graft or growth factors may be mixed in, e.g., the feeding well 510, and endothelial cell layer may be mixed in the culture well 520. In various examples, the cells may migrate to feeding well 510. In another example, in a neurogenesis or neurodegeneration application, growth factors or neurotoxic agents may be mixed to study neurite outgrowth such as described with reference to FIG. 3C discussed above. In studying tumor cell evasion/invasion, migration and wound healing, the migration of cells between the wells such as, e.g., the feeding well 510 or the culture well 520, may be studied. In yet another example, stem cell implantation of fibroblasts to organoids may be studied, as well as T-cell invasion, monocyte migration or other migration assays. In further examples, epithelial and/or mesodermal cell transition processes may be studied, as well as any other known and novel assays including co-culture. In other examples, cardiac beating assays neurospheroids or mini-brain assays with wash may be observed. In yet other examples of multi-tissue toxicity evaluation, interactions in systems of liver-brain, liver-heart, kidney-liver, intestinal-liver, and the like, may be observed and studied via the co-culture microplate arrangements of the present disclosure. In further examples, differentiation and self-renewal of stem cell in response to compounds, toxins, growth factors, inflammation, metabolic switch, may also be observed. Accordingly, various examples of the present disclosure allow for the evaluation of secreted factors, and for the observation of the movement of cells that could be separated by using either horizontal slits, so the cells can migrate, or vertical wider slit for feeding and media-growth factor exchange.

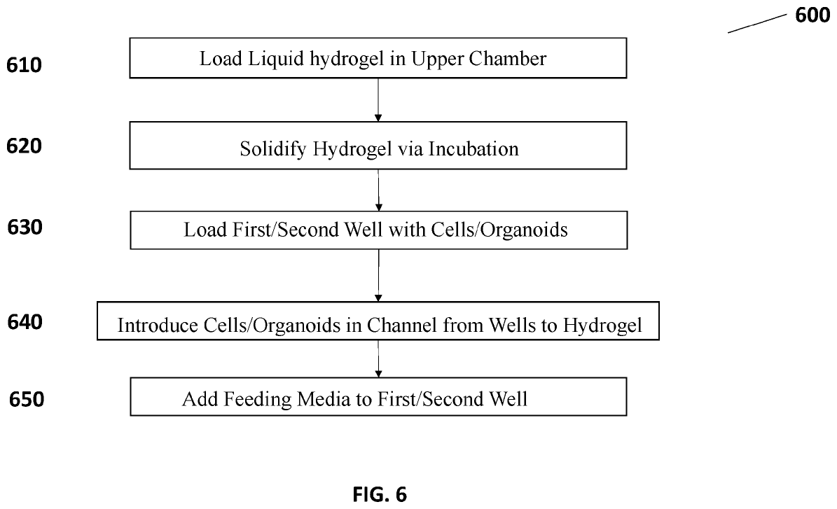

FIG. 6 is a flowchart illustrating a method of separating cells, in accordance with an example of the disclosure. In FIG. 6, the method 600 includes a plurality of operations 610-650 further discussed below. For the sole purpose of convenience, method 600 is described through use of at least the example system 700 described below in combination with the microplate arrangements discussed above. However, it is appreciated that the method 600 may be performed by any suitable system. In FIG. 6, operation 610 includes loading a liquid hydrogel inside the lower chamber of a hydrogel well. For example, the hydrogel well, also referred to herein as a third well, is part of a culture plate that includes a plurality of well plates, each well plate including one or two first wells, one or two second wells, and a channel in fluid communication with the one or two first wells, the one or two second wells, and the third well. As an example, the one or more first wells are feeding wells, the one or more second wells are culture wells. For example, the third well includes an upper chamber and a lower chamber, and during operation 610, the liquid hydrogel is loaded inside the upper chamber. The term “liquid hydrogel” refers to a state of the hydrogel that is close to liquid by that retains its gel nature. During operation 610, loading the liquid hydrogel inside the lower chamber of the hydrogel well includes loading the liquid hydrogel into the upper chamber of the hydrogel well, and transferring the liquid hydrogel form the upper chamber to the lower chamber of the hydrogel well. As another example, during operation 610, the hydrogel well may be loaded with a cell repelling hydrogel so as to, e.g., prevent cells, spheroids or organoids from mixing into the hydrogel. In another example, the hydrogel well may be loaded with a differentiating media or other cells, spheroids and/or organoids. As a further example, loading the hydrogel well may include loading the hydrogel well with epithelial cells. In another example, loading the hydrogel well during operation 610 includes loading a liquid hydrogel premixed with a combination of cells, spheroids and organoids via a loading port in the third well by, e.g., sealingly engaging a pipette with the premixed hydrogel at the loading port.

During operation 620, according to various examples of the disclosure, the method 600 includes solidifying the liquid hydrogel via, e.g., incubation. For example, the culture plate may be maintained at a desired temperature, or at a desired temperature range, for sufficient time to harden the hydrogel. Accordingly, during operation 620, the liquid hydrogel undergoes a phase transformation from substantially liquid to substantially solid. In this case, substantially solid includes a state where the hydrogel is harder than in the liquid phase but retains its gel-like nature characteristics.

During operation 630, according to various examples of the disclosure, the first or second wells are loaded with cells, spheroids and/or organoids. For example, the feeding well and/or the culture well may be loaded with cells, spheroids, organoids and/or tumoroids. In an example, in order to load the feeding well and/or the culture well with cells, spheroids or organoids, the openings of feeding well and/or the culture well may be sealed with, e.g., a pipette, so as to push the air out of the feeding well and/or the culture well prior to loading the cells, spheroids, organoids and/or tumoroids. In various examples, loading the liquid hydrogel during operation 610 and loading the first and second wells during operation 630 may include loading each of the first well, the second well and the hydrogel well with a different combination of cells, spheroids and organoids. As another example, although operation 610 is discussed prior to operation 630, according to various examples, operation 630 may take place, before, after, or contemporaneously with operation 610.

In other examples of the disclosure, the co-culture plate arrangement includes a second channel in fluid communication with the hydrogel well, and also in fluid communication with a fourth well and a fifth well. For example, the fourth well may be another feeding well and the fifth well may be another culture well. In this case, operation 630 includes also loading the fourth well and/or the fifth well with at least one of cells, spheroids or organoids. Operation 630 may also include adding feeding media to any one or more of the first well, the second well, the fourth well and the fifth well. In other examples, any one or more of the first well, the second well, the fourth well and the fifth well may be loaded with cell precursors. As an example, when epithelial cells are loaded in the hydrogel well during operation 610, then the feeding well(s) may be loaded with organoids and/or tumoroids. As a further examples, when the hydrogel well is loaded with a differentiating media, then the feeding well(s) may be loaded with precursor cells. As yet another examples, when the hydrogel well is loaded with a repelling hydrogel, then the culture plate is maintained at a desired temperature to allow the hydrogel to flow to the microchannel. In various examples, loading the liquid hydrogel during operation 610 and loading the first, second, fourth and fifth wells during operation 630 may include loading each of the first well, the second well, the hydrogel or third well, the fourth well and the fifth well with a different combination of cells, spheroids and/or organoids.

During operation 640, cells, spheroids and/or organoids are introduced in the channel, also referred to as microchannel, which is in communication with the feeding well, the hydrogel well and the culture well. In the example where the microplate includes two feeding wells, two culture wells and two microchannels, then during operation 640, cells, spheroids and/or organoids are introduced in both microchannels. For example, the cells, spheroids and/or organoids introduced into both microchannels may be different from each other, where one microchannel may have a type of cells, spheroids and/or organoids and the other microchannel may have a different type of cells, spheroids and/or organoids. In an example, introducing the cells, spheroids and/or organoids into one microchannel, or in two microchannels, includes sealing an opening of the feeding well that is fluidly connected to the microchannel(s) with, e.g., a pipette, and pushing the air out of the microchannel(s) before loading the microchannel(s) with cells, spheroids and/or organoids. As a further example, during operation 640, a hydrogel may be loaded in the first channel and a feeding media may be loaded in the second channel.

During operation 650, in various examples of the present disclosure, feeding media may be added to the feeding well(s) and/or the culture well(s). In various examples, when the feeding media are added, then an interaction may take place between the cells, spheroids and/or organoids present in the channel(s) and the cells introduced in the hydrogel well. For example, because the interface between the hydrogel in the hydrogel well and the media in the channel(s), the media in the channels does not enter the hydrogel well, and the hydrogel and/or media in the hydrogel well does not enter the channel. Accordingly, an interaction may take place at the interface between the microchannel(s) and the hydrogel well. In various examples, this interaction may be observed via openings in the hydrogel well. In other examples, the cells, spheroids and/or organoids that are interacting at the interface between the microchannel(s) and the hydrogel well may be controlled, excised, damaged, and otherwise manipulated so as to observe, e.g., their reaction to various changes or traumas, among other experiments. For example, when neurons are being observed, then the neurons may be intentionally damaged, and the neuron healing or recovery may be observed via the openings in the hydrogel well.

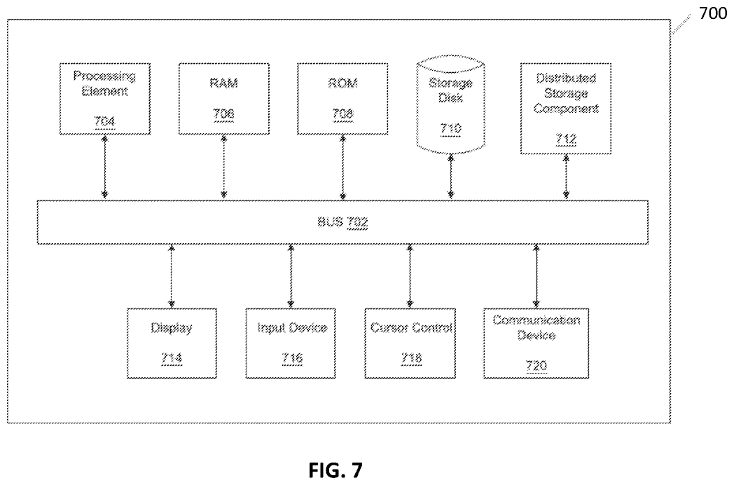

FIG. 7 depicts a block diagram of a computing device, according to various aspects. In the illustrated example, the computing device 700 may include a bus 702 or other communication mechanism of similar function for communicating information, and at least one processing element 704 (collectively referred to as processing element 704) coupled with bus 702 for processing information. As will be appreciated by those skilled in the art, the processing clement 704 may include a plurality of processing elements or cores, which may be packaged as a single processor or in a distributed arrangement. Furthermore, a plurality of virtual processing elements 704 may be included in the computing device 700 to provide the control or management operations for the microplate arrangements 100-500 or the method 600 illustrated above.

The computing device 700 may also include one or more volatile memory(ies) 706, which can for example include random access memory(ies) (RAM) or other dynamic memory component(s), coupled to one or more busses 702 for use by the at least one processing element 704. Computing device 700 may further include static, non-volatile memory(ies) 708, such as read only memory (ROM) or other static memory components, coupled to busses 702 for storing information and instructions for use by the at least one processing element 704. A storage component 710, such as a storage disk or storage memory, may be provided for storing information and instructions for use by the at least one processing element 704. As will be appreciated, the computing device 700 may include a distributed storage component 712, such as a networked disk or other storage resource available to the computing device 700.

The computing device 700 may be coupled to one or more displays 714 for displaying information to a user. Optional user input device(s) 716, such as a keyboard and/or touchscreen, may be coupled to Bus 702 for communicating information and command selections to the at least one processing element 704. An optional cursor control or graphical input device 718, such as a mouse, a trackball or cursor direction keys for communicating graphical user interface information and command selections to the at least one processing element. The computing device 700 may further include an input/output (I/O) component, such as a serial connection, digital connection, network connection, or other input/output component for allowing intercommunication with other computing components and the various components of the microplate arrangements 100-500 or the method 600 illustrated above.

In various embodiments, computing device 700 can be connected to one or more other computer systems via a network to form a networked system. Such networks can for example include one or more private networks or public networks, such as the Internet. In the networked system, one or more computer systems can store and serve the data to other computer systems. The one or more computer systems that store and serve the data can be referred to as servers or the cloud in a cloud computing scenario. The one or more computer systems can include one or more web servers, for example. The other computer systems that send and receive data to and from the servers or the cloud can be referred to as client or cloud devices, for example. Various operations of the microplate arrangements 100-500 or the method 600 illustrated above may be supported by operation of the distributed computing systems.

The computing device 700 may be operative to control operation of the components of the microplate arrangements 100-500 or the method 600 illustrated above through a communication device such as, e.g., communication device 720, and to handle data provided from the data sources as discussed above with respect to the microplate arrangements 100-500 or the method 600. In some examples, analysis results are provided by the computing device 700 in response to the at least one processing element 704 executing instructions contained in memory 706 or 708 and performing operations on the received data items. Execution of instructions contained in memory 706 and/or 708 by the at least one processing element 704 can render the microplate arrangements 100-500 or the method 600 operative to perform methods described herein.

The term “computer-readable medium” as used herein refers to any media that participates in providing instructions to the processing element 704 for execution. Such a medium may take many forms, including but not limited to, non-volatile media, volatile media, and transmission media. Non-volatile media includes, for example, optical or magnetic disks, such as disk storage 710. Volatile media includes dynamic memory, such as memory 706. Transmission media includes coaxial cables, copper wire, and fiber optics, including the wires that include bus 702.

Common forms of computer-readable media or computer program products include, for example, a floppy disk, a flexible disk, hard disk, magnetic tape, or any other magnetic medium, a CD-ROM, digital video disc (DVD), a Blu-ray Disc, any other optical medium, a thumb drive, a memory card, a RAM, PROM, and EPROM, a FLASH-EPROM, any other memory chip or cartridge, or any other tangible medium from which a computer can read.

Various forms of computer readable media may be involved in carrying one or more sequences of one or more instructions to the processing element 704 for execution. For example, the instructions may initially be carried on the magnetic disk of a remote computer. The remote computer can load the instructions into its dynamic memory and send the instructions over a telephone line using a modem. A modem local to computing device 700 can receive the data on the telephone line and use an infra-red transmitter to convert the data to an infra-red signal. An infra-red detector coupled to bus 702 can receive the data carried in the infra-red signal and place the data on bus 702. Bus 702 carries the data to memory 706, from which the processing element 704 retrieves and executes the instructions. The instructions received by memory 706 and/or memory 708 may optionally be stored on storage device 710 either before or after execution by the processing element 704.

In accordance with various embodiments, instructions operative to be executed by a processing element to perform a method are stored on a computer-readable medium. The computer-readable medium can be a device that stores digital information. For example, a computer-readable medium includes a compact disc read-only memory (CD-ROM) as is known in the art for storing software. The computer-readable medium is accessed by a processor suitable for executing instructions configured to be executed.

This disclosure described some examples of the present technology with reference to the accompanying drawings, in which only some of the possible examples were shown. Other aspects can, however, be embodied in many different forms and should not be construed as limited to the examples set forth herein. Rather, these examples were provided so that this disclosure was thorough and complete and fully conveyed the scope of the possible examples to those skilled in the art.

Although specific examples were described herein, the scope of the technology is not limited to those specific examples. One skilled in the art will recognize other examples or improvements that are within the scope of the present technology. Therefore, the specific structure, acts, or media are disclosed only as illustrative examples. Examples according to the technology may also combine elements or components of those that are disclosed in general but not expressly exemplified in combination, unless otherwise stated herein. The scope of the technology is defined by the following claims and any equivalents therein.

1. A culture plate comprising:
an upper surface; and
a plurality of well systems, each well system comprising:
a first well;
a second well;
a first channel in fluid communication with the first well and with the second well; and
a third well disposed between the first well and the second well, wherein the third well is in fluid communication with the first channel, wherein the upper surface of the culture plate defines a well opening for at least one of the first well, the second well, and the third well.

2. The culture plate of claim 1, wherein the first channel is in fluid communication with the first well on a side on the first well and with the second well on a side on the second well.

3. The culture plate of claim 1, wherein the third well comprises:
an upper chamber and a lower chamber having a size less than a size of the upper chamber; and
a first ridge therein configured to receive the first channel;
wherein the first channel is in fluid communication with the lower chamber.

4. The culture plate of claim 1, wherein the first well and the second well are in fluid communication with the first channel via a first opening and a second opening, respectively.

5. The culture plate of claim 1, wherein:
the first channel is a microchannel having an inside diameter of about 5 μm to 100 μm; and/or
an inside diameter of the first channel comprises a protein coating; and/or
a profile of the inside diameter of the first channel is one of circular, oval and polygonal.

6-7. (canceled)

8. The culture plate of claim 1, wherein:
the first channel extends horizontally between the first well and the second well; or
the first channel extends non-linearly between the first well and the second well.

9. (canceled)

10. The culture plate of claim 1, further comprising:
a fourth well;
a fifth well; and
a second channel in fluid communication with the third well, the fourth well and the fifth well;
wherein the third well comprises a second ridge therein configured to receive the second channel.

11. The culture plate of claim 1, wherein the culture plate comprises one of 96 well plates and 348 well plates.

12. A method of cell separation from a media flow in a culture plate comprising a plurality of well plates, each well plate comprising at least a first well, a second well, a third well and a first channel in fluid communication with the first well, the second well and the third well, the third well including an upper chamber and a lower chamber, the method comprising:
loading a liquid hydrogel inside the lower chamber;
solidifying the hydrogel via incubation;
loading at least one of the first well and the second well with at least one of cells, spheroids and organoids;
introducing the at least one of cells, spheroids and organoids in the first channel from the at least one of the first well and the second well to allow an interaction thereof with the hydrogel; and
adding feeding media to the at least one of the first well, the second well and the third well.

13. The method of claim 12, wherein the interaction comprises at least one of cell adherence and organoid formation.

14. The method of claim 12, wherein:
introducing the at least one of cells, spheroids and organoids in the first channel comprises sealing an opening of at least one of the first well and the second well with a pipette; and/or
removing the air comprises one of using a vacuum and sealingly engaging a pipette tip to remove the air from the first channel.

15. The method of claim 12, further comprising at least one of:
accessing at least one of the first well, the second well and the third well via an opening therein to manipulate contents thereof; and
removing air from the first channel before introducing the at least one of cells, spheroids and organoids in the first channel.

16. (canceled)

17. The method of claim 12, wherein loading the hydrogel inside the lower chamber comprises transferring the hydrogel from the upper chamber into the lower chamber.

18. The method of claim 12, wherein the culture plate further comprises a second channel in fluid communication with the third well, a fourth well and a fifth well, the method further comprising:
loading at least one of the fourth well and the fifth well with at least one of cells, spheroids and organoids; and
adding feeding media to at least one of the fourth well and the fifth well.

19. The method of claim 12, further comprising at least one of:
adding epithelial cells to the lower chamber, wherein loading the at least one of the first well and the second well comprises loading the at least one of the first well and the second well with at least one of organoids and tumoroids; and
adding differentiating media to the lower chamber, adding precursor cells to the at least one of the first well and the second well, and imaging neuron healing from the opening in the upper chamber.

20. (canceled)

21. The method of claim 12, wherein loading the liquid hydrogel comprises loading a cell repelling hydrogel, the method further comprising:
maintaining the culture plate at a desired temperature to allow the hydrogel to flow to the first channel before the incubation.

22. The method of claim 12, further comprising at least one of:
filling the first channel with media mixed with cells capable of forming tub vessels in the first channel;
filling the first channel with media mixed with cells capable of forming a lung membrane; and
filling the first channel with media mixed with cells capable of forming a lung membrane, and maintaining the incubation until the cells are settled at a bottom portion of the upper chamber and the lung membrane is formed.

23-24. (canceled)

25. The method of claim 12, wherein:
loading the liquid hydrogel and loading at least one of the first well and the second well comprise loading a different combination of cells, spheroids and organoids in each of the first well, the second well and the third well; and
the first well, the second well and the third well are subsequently fluidly connected by removing air present inside the first channel.

26. (canceled)

27. The method of claim 18, wherein:
loading the liquid hydrogel, loading at least one of the first well and the second well, and loading at least one of the fourth well and the fifth well comprise loading a different combination of cells, spheroids and organoids in each of the first well, the second well, the third well, the fourth well and the fifth well;
the first well, the second well and the third well are fluidly connected by removing air present inside the first channel; and
the third well, the fourth well and the fifth well are fluidly connected by removing air present inside the second channel.

28. The method of claim 12, wherein:
loading the liquid hydrogel comprises loading a liquid hydrogel premixed with at least one of cells, spheroids and organoids in the first channel via a loading port in the third well by sealingly engaging a pipette with the premixed hydrogel at the loading port; or
loading the liquid hydrogel comprises loading a liquid hydrogel premixed with at least one of cells, spheroids and organoids in the first channel and in the second channel via a loading port in the third well by sealingly engaging a pipette with the premixed hydrogel at the loading port.

29. (canceled)
